(19) United States
(12) Patent Application Publication
(10) Pub. No.: US 2025/0375100 A1
(43) Pub. Date: Dec. 11, 2025

(54) GRASPABLE GUIDEWIRE DEVICE

(71) Applicant: Joseph Frederick STAFFETTI, Oldsmar, FL (US)

(72) Inventor: Joseph Frederick STAFFETTI, Oldsmar, FL (US)

(21) Appl. No.: 18/735,767

(22) Filed: Jun. 6, 2024

(51) Int. Cl. A61B 1/01; A61B 1/00; A61B 1/31

(52) U.S. Cl. CPC A61B 1/01; A61B 1/00105; A61B 1/00131; A61B 1/31

(57) ABSTRACT

A graspable guidewire device includes a wire having an indentation portion at its proximal end, a probing portion at its distal end, and a body therebetween. This wire body may be coated in a non-frictive bio-compatible, or otherwise non-reactionary, material that is capable of insertion into a human body without any adverse effects. The probing portion possesses a Young's Modulus that is lower than the body or proximal end of the device while the indentation portion possesses a graspable structure that allows the device to be threaded through an endoscopic instrument. A guide handle is operationally engagable about the indentation portion thereby allowing a medical professional to control the device as it probes deeper into a patient's internal organs. The length of the device is at least twice the distance measured between the tip of a given endoscopic instrument and its accessory (or biopsy) port.

### BACKGROUND

Elongated devices inserted into openings made, or already existing, in a patient's body that maintain against closure or line said openings for further use are well known in the art. A variety of examples of such devices include flexible endoscopic instruments, such as gastroscopes and colonoscopes, endovascular catheters, guidewires, and surgical or trocar cannulae. Typically, these endoscopic instruments are comprised of a handle capable of electronically and/or mechanically controlling an insertion tube having an instrument channel therein, and an accessory or biopsy port allowing insertion of devices into said channel of said tube.

While generally useful, flexible endoscopic instruments and some versions of the other aforementioned devices rely on reaction forces of surrounding bodily tissue within the opening or cavity to control their navigation around corners and bends of internal organs. This approach works well in situations where the internal organs are generally straight, however, if the instrument is not flexible enough then the problems of “looping” and ineffective guiding tend to occur. The former problem, “looping,” occurs when the body of the elongated device is so stiff in relation to the sharp turn of the stiff cavity walls that the device's distal end curls back into its own pathway thereby creating a “loop” within the patient leading to discomfort and/or pain wherein continuing to probe may simply increase the size of the single loop or create more loops thereby causing further patient distress and/or damage to the cavity walls. This problem of looping tends to occur more in internal organs having a large amount of space between the guiding walls, like the stomach, or organs having tight corners along their tortuous and circuitous walled pathways, like the colon or small intestines. Should looping occur, operators currently use a series of maneuvers that may include pushing, pulling, rotating, jiggling, inflation/deflation (depending on the device), external abdominal compression, and/or changing of the patient's position, however, these may cause additional discomfort while allowing the sigmoid colon to reform if the instrument is retracted too much when seeking to alleviate the patient's pain/discomfort. The latter problem, ineffective guiding, typically occurs when an elongated device is not flexible enough to allow for proper guidance into branches or specific regions of an internal organ or body cavity, which may be due to the pathway being too sharply angled or tortuous and/or the device having increased stiffness, low maneuverability, unsuitable dimensions, or some combination thereof.

Specifically, traversing the tortuous sigmoid colon tends to be especially difficult for endoscopy and colonoscopy instruments as well as for guidewires, catheters, cannulae, and equivalent devices. This is due to the tight turn that any instrument must make to traverse beyond the sigmoid colon before entering the relatively straighter pathway of the remaining colon. For that reason, doctors performing colonoscopies May prefer at times to use gastroscopes, which have a significantly shorter insertion tube than colonoscopes, in order to traverse beyond the sigmoid colon due to its slimmer and more flexible insertion tube. However, once the gastroscope has advanced beyond the tight curves of the sigmoid colon it is unable to explore any further with its given length and is therefore unable to reach the cecum portion of the colon, which is furthest away from the rectum and is a common location for cancer.

Therefore, an additional device must then be inserted into the patient's internal organs through the gastroscope's instrument channel to maintain the position and pathway of the gastroscope through the sigmoid colon after it is removed. Only then can an additional device, such as an endoscope, colonoscope, or equivalent instrument traverse the now straightened out pathway through and beyond the sigmoid colon with minimal interference and maneuvering required. However, problems exist with how best to introduce the secondary endoscopic instrument to advance beyond the sigmoid colon and occupy the position of the replaced gastroscope without allowing the sigmoid colon to reform. One such method is to use an additional device. It should be noted that such an additional device, such as a guidewire or catheter, can't simply be introduced into the tip of a secondary endoscopic instrument through the instrument channel and then advanced because, if it did so, it would not be capable of exiting through the accessory (or biopsy) port due to the structure of the instrument channel. In order to pull the guidewire through the instrument channel and out of the accessory (or biopsy) port, it must be grasped by another device, such as a snare.

Previous solutions to the aforementioned problems relating to the use of endoscopic instruments, specifically pertaining to the traversal of the colon during colonoscopies, may include the introduction of sigmoid splints, overtubes (wherein central cores thereof may be separable or removable), conversely rigidizing and relaxing cannulae utilizing incremental interlocking connectors, pneumatic or vacuum pressure alterations (or seals), as well as motorized means of controlling the distal tip of these or equivalent devices. Additional solutions contemplated in the field relate to versions of the aforementioned devices that engage to the exterior of the endoscope, or other endoscopic instrument, and assist in guiding its movement and maneuvering.

It is understood that operators and medical professionals still face significant difficulty in inserting and advancing an endoscopic instrument throughout the colon, specifically beyond the sigmoid colon, without the occurrence of looping or situations that cause discomfort to the patient. As a result, what is needed is a device that improves the use of said endoscopic instruments, that simplifies procedures relating to exploration of a patient's colon (and potentially other internal organs), and that reduces the requisite effort and potential consternation of the medical professional conducting the procedure as well as the likelihood of patient discomfort therefrom. This device should be relatively inexpensive to produce, able to be disinfected, unlikely to cause negative bodily reactions, as well as capable of being easily learned then used by medical professionals with a higher probability of success than alternatives.

### FIELD OF THE ART

The present invention relates to a medical guidewire, and more particularly, to a graspable guidewire that may be used in conjunction with existing endoscopic instruments, such as a gastroscope or colonoscope, in order to navigate internal organs, particularly the colon or large intestine.

### SUMMARY

The general purpose of the graspable guidewire device, described subsequently in greater detail, is to provide a graspable guidewire device for medical use in conjunction with existing endoscopic instruments that has novel features that is not anticipated, rendered obvious, suggested, or even implied by prior art, either alone or in combination thereof.

According to one embodiment of the graspable guidewire device, medical professionals tend to have difficulty traversing the sigmoid colon portion during colonoscopies due to its marked tortuosity and angulation in combination with the limitations of the colonoscope (or endoscope) instrument. As stated above, the operator will instead insert a slimmer and more flexible instrument, such as a gastroscope, into the patient and beyond the problematic sigmoid colon section but no further given this scope's considerably shorter insertion tube. This forecloses the ability to view the entirety of the colon, such as the cecum whereat cancer is known to form.

While versions of the aforementioned apparatuses may offer various solutions to this problem, these devices are likely expensive to produce and require considerable training to operate. The graspable guidewire device is capable of being inserted into the instrument channel of the gastroscope (or equivalent placeholder instrument), thereby maintaining the straightened pathway through, and position beyond, the sigmoid colon while the gastroscope instrument is removed. The graspable guidewire device then simply requires a grasping element (such as a snare) disposed through a secondary endoscopic instrument (such as a longer endoscope, colonoscope, or equivalent instrument) to easily and simply operationally engage the proximal end of the device via its indentation portion. Once the snare disposed through the secondary endoscopic instrument has operationally engaged the guidewire device, it may be pulled through the accessory (or biopsy) port of the secondary endoscopic instrument such that said instrument's insertion tube may be advanced over the wire through and beyond the sigmoid colon without causing pain or discomfort to the patient and while also avoiding any problematic looping.

The illustrated embodiment of the graspable guidewire device, as well as other embodiments contemplated herein, is dimensioned to allow for the repeated insertion and removal of the device into and out of existing endoscopic instruments via their instrument channels and accessory (or biopsy) ports. In this way, embodiments of the graspable guidewire device may be capable of being inserted into the initial endoscopic instrument, such as a gastroscope, in order to preserve the location of the distal end thereof beyond the sigmoid colon, while also allowing for the exchange of endoscopic instruments by enveloping and then removing said graspable guidewire device. Alternatively, the graspable guidewire device may remain present within and beyond the enveloping endoscopic instrument's tube within the patient thereby continuing to perform as a probing guide. Embodiments of the graspable guidewire device are contemplated as including markings, of varying colors and shapes and designs, along at least one circumference of its body which will quickly inform the operator of the length of device remaining when inserting, transitioning, and probing with the device.

Unlike previously disclosed guidewires and catheters, the indentation portion at the proximal end of the graspable guidewire device allows for quick and simple operational engagement thereto via a snare, hook, or equivalent grasping element. Once the graspable guidewire device has been pulled through the secondary endoscope's instrument channel and out of its accessory (or biopsy) port, and the instrument is advanced into the proximal part of the colon, the device is capable of being entirely removed from both the patient and endoscopic instrument via said channel and port. Alternatively, embodiments are contemplated herein as including a securably removable guide handle that may be releasably fastenable about the proximal end of the graspable guidewire device. This may occur absent the presence of an enveloping endoscopic instrument or once the proximal end of the graspable guidewire device has exited the endoscope's accessory (or biopsy) port, but before the distal end has exited the patient, to allow for more control by the medical professional as they probe further into a patient's internal organs. Said securably removable guide handle is contemplated as embodying a variety of shapes, designs, and formations.

It is additionally contemplated that embodiments of the graspable guidewire device may include markings of various designs, shapes, and colors along or around its body corresponding to lengths or depths such that operators need only view a portion of the device to know how much further the device may probe. Further, these markings are contemplated as being able to denote lengths of the device's body alone as well as lengths of the device as it may relate to an endoscopic instrument's dimensions and components.

As illustrated herein, a contemplated embodiment of the graspable guidewire device includes a probing portion tapered to a relatively flexible tip at its distal end although additionally contemplated embodiments may include probing portions that appear to simply be extensions of the device's body, with substantially similar dimensions, as well as tapered designs having rounded, bulbous, U-shaped, or alternatively shaped tips. These alternative probing portion tips, of various embodiments, are contemplated as being more flexible than the body of the graspable guidewire device, such that the Young's Modulus of the body is greater than that of the distal end. The increased flexibility of the distal end will allow for flexing or bending of the distal end's probing portion as it maneuvers through, and is turned by, the relatively stiffer walls of a patient's internal organs, as opposed to puncturing those walls, while the stiffness of the guidewire's body may allow the device to maintain the straightened pathway that is created.

An embodiment of the graspable guidewire device is contemplated, and depicted herein, as possessing an indentation portion resembling a rounded hook extending from the body at its proximal end. The majority of embodiments contemplated herein include a non-sharp proximal end and indentation portion such that no damage will be caused to the device itself or any existing endoscopic instrument when it enters, is pulled through, and exits endoscopic instrument's instrument channel and accessory (or biopsy) port. Additional contemplated embodiments of the indentation portion of the graspable guidewire may include at least one notch, groove, hook, slot, or equivalent structural element capable of being grasped as well as capable of being contained within a securably removable guide handle component.

The guide handle component is contemplated and depicted in the present embodiment as possessing a hinged element and a releasably fastenable element, such as snaps, which allow it to encompass and close over the proximal end of the graspable guidewire device. However, additionally contemplated embodiments of the graspable guidewire device may forgo the hinged element in lieu of alternative functionally equivalent components and/or may replace the snaps with alternative releasably fastenable elements, such as magnets, clips, latches, clasps, or equivalent components. Separately contemplated embodiments of the guide handle may include a channel spanning the full length of said handle within which a portion of the graspable guidewire device's body may be disposed and enclosed, as opposed to the proximal end, such that the device will extend outward from two sides of the handle. These contemplated embodiments would give operators the ability to handle the guidewire device from a position more proximal to the patient. However, to counter the likelihood of sliding either by the device's body through the handle and/or the handle along the body, it is further contemplated that at least one indentation, groove, rib, bump, ring, or equivalent element be present along the body with its complementary shape present within the handle's channel such that closing the handle once these complementary portions are aligned will assure no slippage or sliding during use. It is important to note that while these additionally contemplated components may either increase or decrease the width or circumference of the body, they will not alter the structural stability or integrity of the device.

Thus has been broadly outlined the more important features of the graspable guidewire device so that the detailed description thereof that follows may be better understood and in order that the present contribution to the art may be better appreciated. For better understanding of the graspable guidewire device, its operating advantages and specific objects attained by its uses, refer to the accompanying drawings and description.

### BRIEF DESCRIPTION OF THE DRAWINGS FIGURES

FIGS. 1A through 1C depict example embodiments of the graspable guidewire device's indentation portion.

FIGS. 2A through 2C depict example embodiments of the graspable guidewire device's probing portion.

FIGS. 3A through 3D depict side, top, and front views of an example embodiment of the securably removable guide handle.

FIG. 4 is a front view of an example embodiment of the graspable guidewire device disposed within a patient's internal organs.

FIG. 5 is an isometric view of a snare instrument disposed through an endoscopic instrument operationally coupled to an example embodiment of the indentation portion of the graspable guidewire device.

FIG. 6 is an isometric view of an endoscopic instrument with an example embodiment of the graspable guidewire device disposed therethrough.

FIG. 7 is a front view of an endoscopic instrument enveloping and being guided by the same graspable guidewire device embodiment through a patient's internal organs.

FIGS. 8A and 8B are isometric views of a cross-section of the graspable guidewire device's wire enclosed in a coating to form the body with example embodiments of markings included thereon.

### DETAILED DESCRIPTION OF THE DRAWINGS

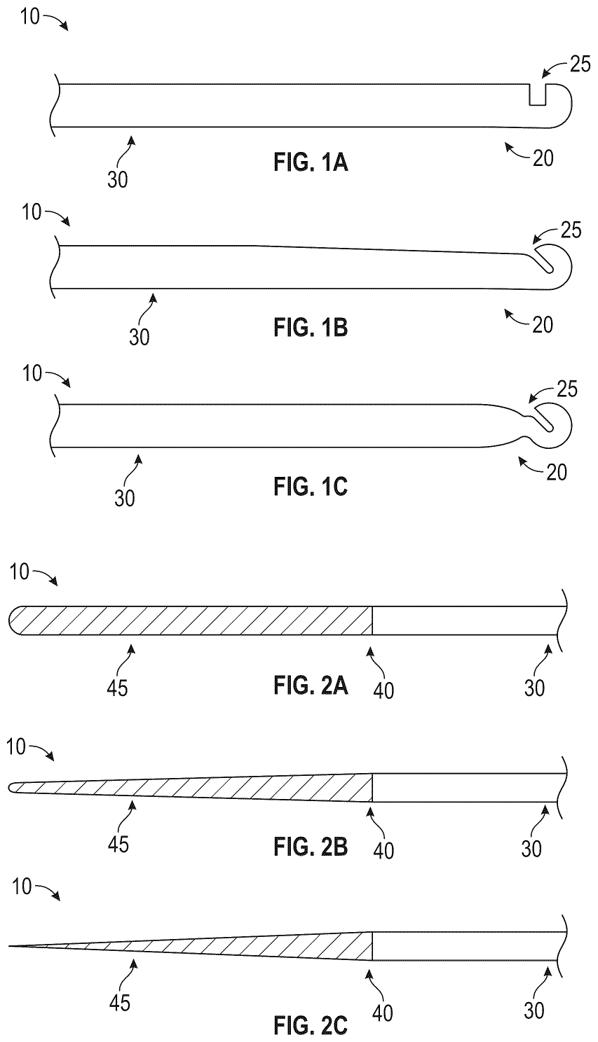

With reference now to the drawings, and in particular FIGS. 1A through 8B thereof, examples of graspable guidewire device employing the aforementioned principles and concepts, generally designated by the reference number 10, will be described.

Referring to FIGS. 1A through 1C, a few of the various contemplated embodiments of indentation portion 25 disposed about the proximal end 20 of graspable guidewire device 10 are illustrated. While there are a variety of potential shapes and forms that indentation portion 25 may embody, FIG. 1A depicts a uniform diameter relative to body 30 with a square slot, FIG. 1B depicts a uniform diameter relative to body 30 with an angled rounded slot, and FIG. 1C depicts a bottlenecked diameter expanding into a angled rounded slot.

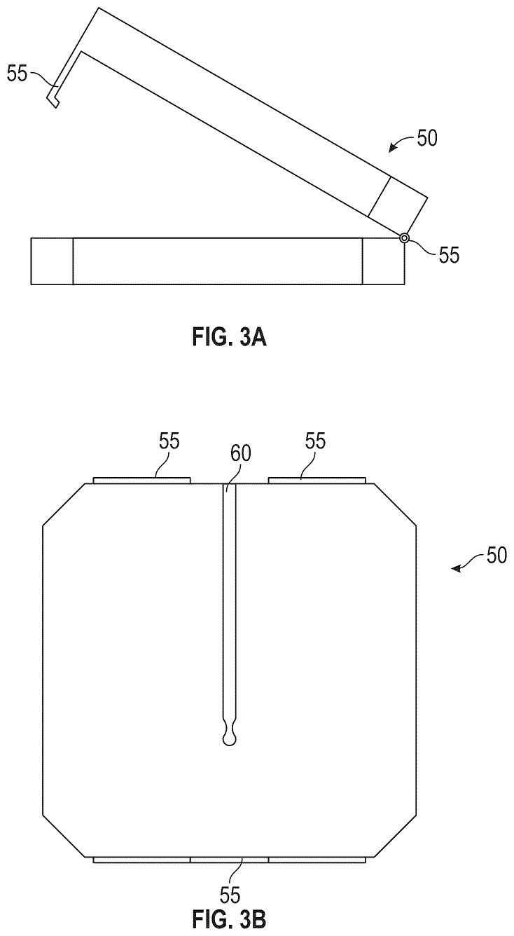

Referring next to FIGS. 2A through 20, a few of the various contemplated embodiments of probing portion 45 disposed about distal end 40 of graspable guidewire device 10 are illustrated. While there are a variety of potential shapes and forms that probing portion 45 may embody, FIG. 2A depicts a uniform diameter relative to body 30 with a rounded tip, FIG. 2B depicts a tapering diameter relative to body 30 with a rounded tip, and FIG. 2C depicts a tapering diameter relative to body 30 with a pointed tip. It should be noted that in at least the contemplated embodiments illustrated, probing portion 45 possesses a Young's Modulus lower than that of body 30 and proximal end 20 such that probing portion 45 is more flexible than the remaining sections of graspable guidewire device 10. The decreased Young's Modulus of probing portion 45 is contemplated either as being uniform along the length of said portion or gradually decreasing along said portion's length such that the most flexible area is at the most distal tip.

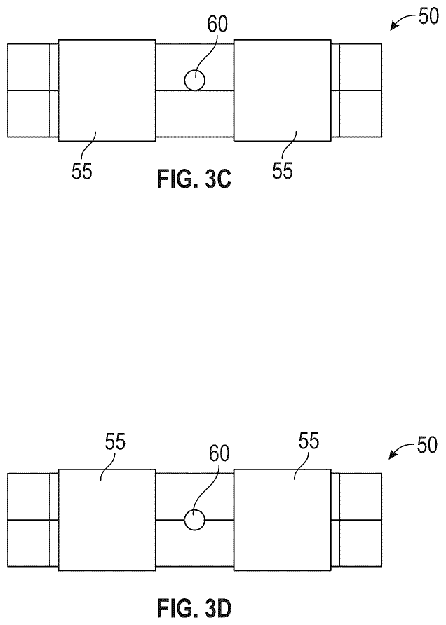

Referring next to FIGS. 3A through 3D, side, top, and front views depict a contemplated example embodiment of securably removable guide handle 50. As illustrated in these figures, guide handle 50 possesses hinged fastenable element 55 on its rear and clip elements on its front side such that its top and bottom may open while remaining connected to one another. Additionally contemplated embodiments of guide handle 50 may possess alternative fastenable elements 55 that effectuate the same or similar functionality, such as snaps, clasps, pins, hinges, and other equivalent structural elements, as well as magnetic elements inlayed within each guide handle 50 portion to allow for simple and repeatable opening and closing without a need for hinges. Once opened, indentation portion 25 and a section of proximal end 20 of graspable guidewire device 10 are housed within channel 60, designed as a complementary molded space formed in at least one side of guide handle 50. This channel 60 may be present in just one half of guide handle 50, or may be partially present in both, such that proximal end 20 and indentation portion 25 remain stationary once enclosed therewithin. As aforementioned, additionally contemplated embodiments may include channel 60 spanning the distance of one, or both, guide handle 50 halves such that body 30 of graspable guidewire device 10 may lay and be contained within guide handle 50. Further, it is contemplated that these embodiments possessing full-length channel 60 may also include complementary indentations, grooves, ribs, bumps, rings, or equivalent structural alterations along body 30 that align with complementary shapes, designs, and molded spaces of channel 60 within guide handle 50 such that once enclosed about graspable guidewire device 10, there will be no sliding or moving along body 30 while in use.

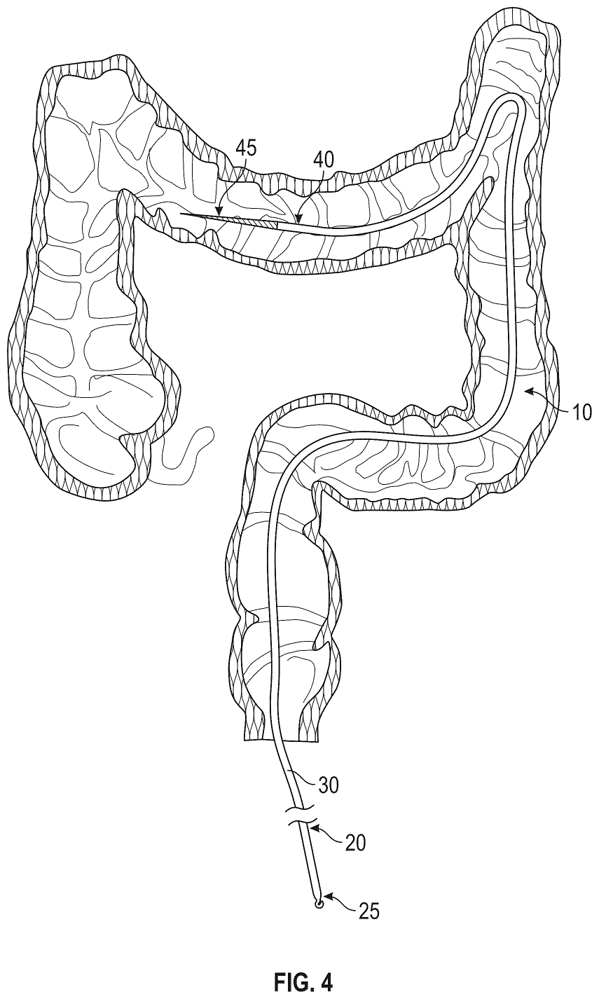

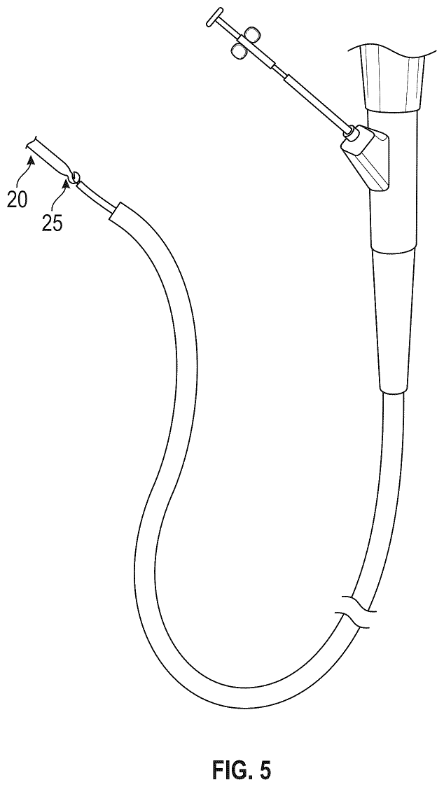

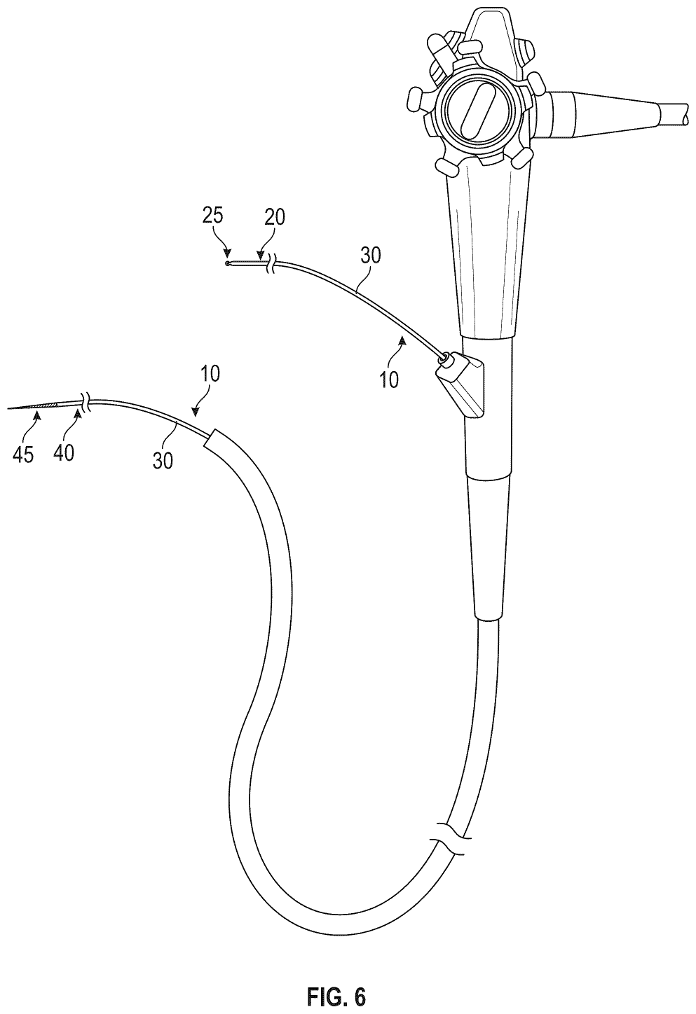

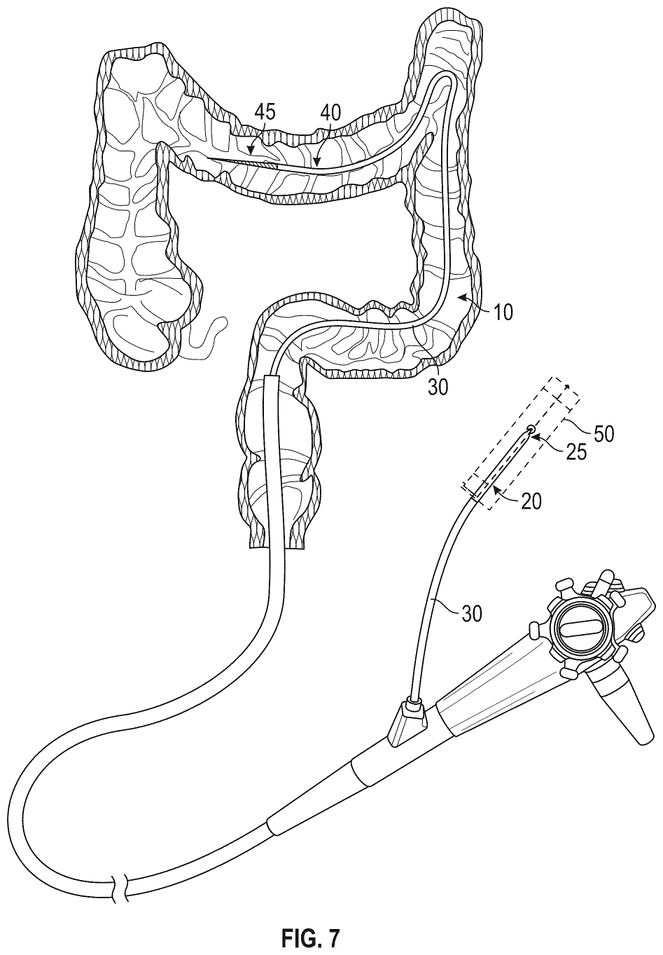

Referring next to FIGS. 4 through 7, which depict the contemplated process of disposing graspable guidewire device 10 through an endoscopic instrument, a view of a user's internal organs having graspable guidewire device 10 disposed therein, and an endoscopic instrument enveloping graspable guidewire device 10 via a snare instrument disposed therethrough thereby using it as a guide to progress deeper into a patient. After a first endoscopic instrument has been inserted into a patient's colon, such as a gastroscope that is slim and flexible, but lacks the length to traverse the entire colon, graspable guidewire device 10 may be inserted through the endoscopic instrument's accessory (or biopsy) port and into the instrument channel. As depicted in FIG. 4, graspable guidewire device 10 may be used to progress further into a patient's internal organs after the first endoscopic instrument is removed, thereby leaving the majority of the length of graspable guidewire device 10 in place while indentation portion 25 of proximal end 20 remains exterior to the patient. In FIG. 5, a snare instrument is disposed through the accessory (or biopsy) port and through the instrument channel of a second endoscopic instrument to operationally couple to indentation portion 25 of proximal end 20 of graspable guidewire device 10. As depicted in FIG. 6, the snare instrument has successfully pulled graspable guidewire device 10 through the instrument channel and accessory (or biopsy) port of said second endoscopic instrument, thereby allowing said instrument to advance over the length of graspable guidewire device 10 maintained within the patient such that there are minimal issues relating to pain, discomfort, or looping as well as a higher probability of a successful operation, as depicted in FIG. 7.

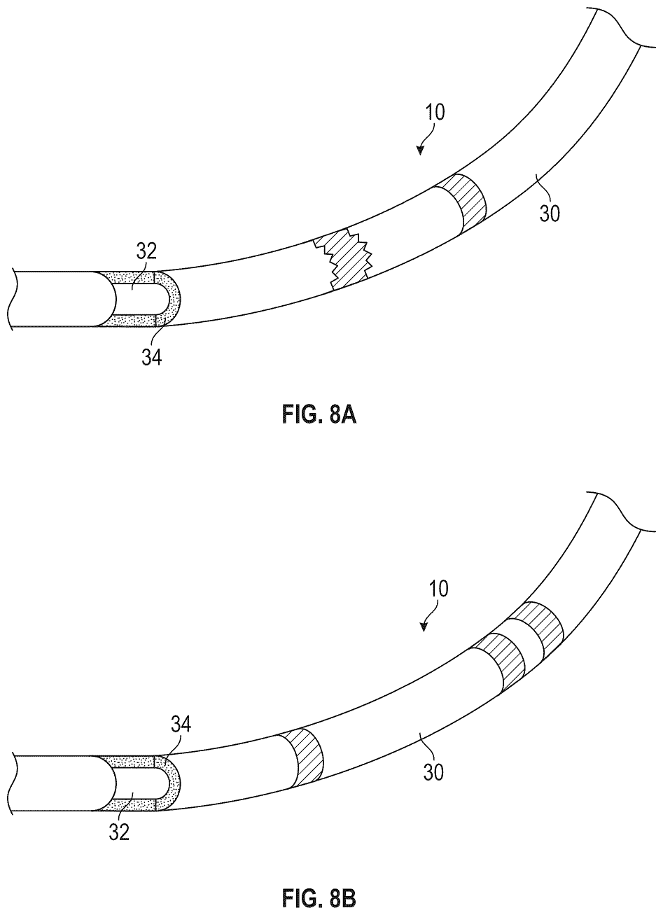

Referring lastly to FIGS. 8A and 8B, a section of body 30 is depicted as having wire 32 enclosed by coating 34. In the present illustration, coating 34 is contemplated as comprising a non-frictive biocompatible polymeric material whilst wire 32 is contemplated herein as comprising a rigid metallic material. However, additionally contemplated example embodiments may include wire 32 as comprising a less rigid material, such as various polymers and bio-plastics as well as biocompatible materials, capable of similarly maintaining the straightened pathway created by the use of graspable guidewire device 10, while further contemplated example embodiments of coating 34 may include various materials that do not cause reactions (adverse or otherwise) to a patient's internal organs, such as bio-plastics and certain metals or metallic alloys. Moreover, contemplated embodiments may include various markings along body 30 that show operators the remaining and/or used length of graspable guidewire device 10 thereby informing their decision to continue probing or begin retraction. Said markings may include various colors, shapes, designs, and numbers thereof and are contemplated as being based either on the length of graspable guidewire device 10 itself or in relation to the lengths of various endoscopic instruments and/or components thereof. These markings, similar to the aforementioned contemplated bodily grooves, are not intended to impact, alter, or affect the structural integrity of the present invention nor contemplated embodiments thereof.

1. A graspable guidewire device comprising:
a wire comprising:
a proximal end;
a distal end; and
a body therebetween;
an indentation portion disposed about the proximal end; and
a probing portion disposed at the tip of the distal end;
wherein the indentation portion is graspable by or otherwise operationally couplable to a snare, handle, forceps, or equivalent instrument component.

2. The graspable guidewire device of claim 1 further comprising a non-frictive biocompatible coating enclosing the wire.

3. The graspable guidewire device of claim 2 wherein the wire has a Young's Modulus that is lower at the distal end than that of the body and proximal end.

4. The graspable guidewire device of claim 3 wherein said Young's Modulus decreases along the distal end to a minimum at the probing portion.

5. The graspable guidewire device of claim 3 wherein said Young's Modulus is uniform along the length of the distal end.

6. The graspable guidewire device of claim 3 wherein the indentation portion is a notch, hook, groove, slot, or equivalent structure.

7. The graspable guidewire device of claim 6 further comprising a securably removable guide handle operationally engaged about the indentation portion.

8. The graspable guidewire device of claim 7 wherein the securably removable guide handle includes a channel in which at least the indentation portion will lay.

9. The graspable guidewire device of claim 8 wherein the securably removable guide handle is releasably securable about the proximal end via fastenable elements.

10. The graspable guidewire device of claim 6 wherein the distal end tapers to a tip having a minimum diameter not less than 0.035 inches.

11. The graspable guidewire device of claim 6 wherein the wire dimensions permit it to enter and exit the accessory (or biopsy) port and instrument channel of an existing endoscopic instrument or equivalent instrument.

12. The graspable guidewire device of claim 11 wherein the wire is long enough to traverse at least the sigmoid colon of a patient while an adequate length remains exterior to the patient for said operational coupling of the indentation portion.

13. The graspable guidewire device of claim 12 wherein an adequate length of the wire is more than twice the distance measured between the tip of the endoscopic instrument and the instrument's accessory (or biopsy) port.

14. The graspable guidewire device of claim 2 wherein the non-frictive biocompatible coating is polymeric or formed of a bio-plastic.

15. A graspable guidewire device comprising:
a wire comprising:
a proximal end;
a distal end; and
a body therebetween;
a non-frictive biocompatible coating enclosing the wire;
an indentation portion disposed about the proximal end;
a removable guide handle releasably securable about the indentation portion; and
a probing portion disposed at the tip of the distal end;
wherein the indentation portion is graspable by or otherwise operationally couplable to a snare, handle, forceps, or equivalent instrument component.

16. The graspable guidewire device of claim 15 wherein the wire has a Young's Modulus that is lower at the distal end than that of the body and proximal end.

17. The graspable guidewire device of claim 16 wherein the indentation portion is a notch, hook, groove, slot, or equivalent structure.

18. The graspable guidewire device of claim 17 wherein the removable guide handle is releasably securable via fastenable elements.

19. The graspable guidewire device of claim 18 wherein the wire not housed within the removable guide handle is long enough to traverse at least the sigmoid colon of a patient thereby permitting the running of an existing endoscopic instrument, or equivalent instrument, along the guided pathway therethrough.

20. The graspable guidewire device of claim 15 wherein the non-frictive biocompatible coating is polymeric or formed of a bio-plastic.
